(19) United States
(12) Patent Application Publication
(10) Pub. No.: US 2023/0230249 A1
(43) Pub. Date: Jul. 20, 2023

(54) DIGITAL ANTIMICROBIAL SUSCEPTIBILITY TESTING

(71) Applicant: ARIZONA BOARD OF REGENTS ON BEHALF OF ARIZONA STATE UNIVERSITY, Scottsdale, AZ (US)

(72) Inventors: Shaopeng WANG, Chandler, AZ (US); Fenni ZHANG, Tempe, AZ (US); Nongjian TAO, Fountain Hills, AZ (US)

(73) Assignee: ARIZONA BOARD OF REGENTS ON BEHALF OF ARIZONA STATE UNIVERSITY, Scottsdale, AZ (US)

(21) Appl. No.: 18/002,438

(22) PCT Filed: Jun. 23, 2021
(86) PCT No.: PCT/US2021/038750

§ 371 Date: Dec. 19, 2022

Related U.S. Application Data
(60) Provisional application No. 63/043,713, filed on Jun. 24, 2020.

(51) Int. Cl. G06T 7/00; C12Q 1/06; C12Q 1/18; C12M 1/00

(52) U.S. Cl. CPC G06T 7/0016; C12Q 1/06; C12Q 1/18; C12M 41/06

(57) ABSTRACT

Detecting single bacterial cells in a sample includes collecting, from a sample provided to an imaging apparatus, a multiplicity of images of the sample over a length of time; assessing a trajectory of each bacterial cell in the sample; and assessing, based on the trajectory of each bacterial cell in the sample, a number of bacterial cell divisions that occur in the sample during the length of time.

### CROSS-REFERENCE TO RELATED APPLICATION

This application claims the benefit of U.S. Patent Application No. 63/043,713 entitled “DIGITAL ANTIMICROBIAL SUSCEPTIBILITY TESTING” and filed on Jun. 24, 2020.

### STATEMENT OF GOVERNMENT INTEREST

This invention was made with government support under R01 AI138993 awarded by the National Institutes of Health. The government has certain rights in the invention.

### TECHNICAL FIELD

This invention relates devices, systems, and methods for rapid digital antimicrobial susceptibility testing by imaging and tracking single cells in a clinical sample.

### BACKGROUND

Antimicrobial resistance is a rapidly growing threat to global public health, affecting millions of people annually. One cause for this global concern is the misuse and overuse of antimicrobials. Antimicrobial susceptibility testing (AST) methods typically used in clinical labs rely on overnight cell culture for pathogen infection detection and can involve additional isolation and sub-culture steps. Various emerging AST technologies have been proposed, which fall into two categories: genotypic and phenotypic approaches. Genotypic approaches can be used to detect antibiotic resistance genes. While sensitive, this approach requires prior knowledge of the pathogens, which can lead to false negatives when a new resistant strain emerges, and false positives because resistance genes do not necessarily produce resistant strains. Phenotypic approaches measure a phenotypic feature, such as size or number of bacterial cells. However, most AST technologies still require culture, isolation, and enrichment of bacterial cells.

For phenotypic AST, a common practice is to compare the bacterial cell growth in samples with and without antibiotics. In some cases, morphology changes (e.g., cell size), DNA/RNA copy changes, or cell number changes are used to quantify cell growth. However, for rapid AST with real samples, some features are not reliable. For example, bacteria continue growing in DNA and size at the initial phase with the presence of some antibiotics, and particulate contaminates can interfere with the DNA/cell number counting methods.

### SUMMARY

This disclosure relates systems and methods for rapid antimicrobial susceptibility testing (AST) using large volume scattering imaging (LVSi) for culture-free, rapid imaging and tracking single cells in clinical samples, including clinical samples with low bacterial counts. Single cell division events are tracked, allowing rapid identification of viable bacterial cells in the samples and AST without cell culturing. Single cell division measures the growth of live cells only, and is generally not sensitive to other impurities (e.g. crystals, cell debris, or dead bacterial cells).

In a first general aspect, detecting single bacterial cells in a sample includes collecting, from a sample provided to an imaging apparatus, a multiplicity of images of the sample over a length of time; assessing a trajectory of each bacterial cell in the sample; and assessing, based on the trajectory of each bacterial cell in the sample, a number of bacterial cell divisions that occur in the sample during the length of time.

Implementations of the first general aspect may include one or more of the following features.

The first general aspect may further include providing the sample to the imaging apparatus, collecting the sample from a subject, or both. The sample may be a bodily fluid (e.g., urine) from a subject. In some cases, the first general aspect further includes combining the sample with a culture medium. In certain cases, the first general aspect may further include diluting the sample, filtering the sample, or both.

In some implementations, the first general aspect further includes defining an infection threshold as a number of cell divisions, and comparing the number of bacterial cell divisions that occur in the sample during the length of time with the infection threshold. Some implementations further include identifying the sample as infection positive if the number of bacterial cell divisions that occur in the sample during the length of time exceeds the infection threshold or identifying the sample as infection negative if the infection threshold exceeds the number of bacterial cell divisions that occur in the sample during the length of time. In one example, the infection threshold is between 2 to 10 cell divisions. The length of time is typically in a range between 20 minutes and 120 minutes, or between 30 minutes and 60 minutes.

In some implementations, the sample is a first sample, the length of time is a first length of time, and the number of bacterial cell divisions that occur in the sample during the first length of time is a first number of bacterial cell divisions, and the first general aspect further includes collecting, from a second sample provided to the imaging apparatus, a multiplicity of images of the second sample over a second length of time; assessing a trajectory of each bacterial cell in the second sample; and assessing, based on the trajectory of each bacterial cell in the second sample, a second number of bacterial cell divisions that occur in the second sample during the second length of time. The first sample and the second sample may be obtained from a common source. In some cases, the first sample includes an antibiotic and the second sample is free of added antibiotic. Some implementations further include assessing a ratio of the first number of bacterial cell divisions to the second number of bacterial cell divisions. Some implementations further include defining a susceptibility threshold and comparing the ratio to the susceptibility threshold. Certain implementations further include identifying the first sample as resistant to the antibiotic if the susceptibility ratio exceeds the threshold or identifying the first sample as susceptible to the antibiotic if the susceptibility threshold exceeds the ratio. In some cases, the susceptibility threshold is in a range of 0.4 to 0.6, corresponding to inhibition of 40% to 60% of the bacterial cells, respectively.

In some implementations, a volume of the sample is in a range of 1 μL to 50 μL. A number of particles in the sample is typically less than about 2×10⁵ particles/mL.

In some implementations, assessing the trajectory of each bacterial cell in the sample includes monitoring a position of each bacterial cell in a sequence of images. A magnification of the imaging apparatus is typically in a range of 0.5-10×.

Collecting the multiplicity of images may include irradiating the sample with light (e.g., infrared light). The sample may be a liquid sample. The sample is typically uncultured.

The first general aspect may further include, based on the number of bacterial cell divisions, administering an antibiotic to a subject (e.g., a mammalian subject or a human).

In a second general aspect, a system includes a light source, optics configured to focus light from the light source on a liquid sample in a container, an imaging device, and a controller operably coupled to the light source and the imaging device and configured to initiate collection of a series of images of the liquid sample over a length of time. Based on the images, the controller is further configured to assess a trajectory of each bacterial cell in the sample and to assess, based on the trajectory of each bacterial cell in the sample, a number of bacterial cell divisions that occur in the sample during the length of time.

In the AST systems and methods described, single cell sensitivity is achieved without immobilization or further processing (e.g., enrichment or culturing) of cells. Large sample volumes can be used in cuvettes or vials without additional reagents (e.g., DNA primers, enzymes, binding agents, etc.) or microfluidics. Results can be achieved within one hour with 97% accuracy, allowing precise antibiotic prescription and timely treatment of patients during clinic visits.

The described AST systems and methods overcome difficulties with traditional methods such as optical microscopy that can image bacterial cells but typically require immobilization of the cells on a surface. This feature of traditional optical microscopy, together with the small field of view of high-resolution optical microscopy, makes it difficult to locate bacterial cells in low concentration samples without enrichment. LVSi overcomes this difficulty by illuminating and imaging a large sample volume such that the presence of a few bacterial cells in a clinical sample can be tracked continuously.

The details of one or more embodiments of the subject matter of this disclosure are set forth in the accompanying drawings and the description. Other features, aspects, and advantages of the subject matter will become apparent from the description, the drawings, and the claims.

### BRIEF DESCRIPTION OF DRAWINGS

FIG. 1A depicts an experimental setup for large volume scattering imaging (LVSi) of a clinical urine sample. FIG. 1B shows single cell trajectory tracking. FIG. 1C shows single cell division tracking examples for digital antibiotic susceptibility testing (AST). FIG. 1D shows clinical decision determination based on detected division events.

FIG. 2 is a flow chart showing a process for assessing cell division events.

FIG. 3 shows division over-counting with different cell numbers (n=3).

FIGS. 4A-4F show various aspects of LVSi analysis of antibiotic susceptible E. coli. FIGS. 4A and 4D show snapshots of cell images at different time points with and without antibiotics, respectively. FIGS. 4B and 4E show tracked division events during a video at each time point with and without antibiotics, respectively. FIGS. 4C and 4F show cumulative division events over time with and without antibiotics, respectively.

FIGS. 5A-5D show statistical results of the division tracking for digital AST with different antibiotics. FIGS. 5A and 5C show representative cumulative division over time plots for E. coli with and without antibiotics. FIGS. 5B and 5D show statistical analysis of bacterial growth at different time points with and without antibiotics.

FIGS. 6A-6G show rapid LVSi infection detection with 60 clinical urine samples. FIG. 6A shows representative division tracking result of one infection negative sample and one infection positive sample. FIG. 6B shows cumulative division results over 60 min of 30 infection negative clinical samples. FIG. 6C shows normalized cumulative division results (normalized by the cumulative division events at 60 min) over 60 min of 30 infection positive clinical samples. FIGS. 6D-6F show the comparison of a reference method and digital AST determinations of infection at 30 min, 45 min, and 60 min, respectively. FIG. 6G shows the infection detection accuracy over detection time.

FIGS. 7A-7H show results of digital AST with infection positive clinical samples. FIG. 7A shows a representative single-cell division tracking result of a susceptible sample.

FIG. 7B shows a representative single-cell division tracking result of a resistant sample. FIG. 7C shows normalized cumulative division counting results of all 22 susceptible samples. FIG. 7D shows normalized cumulative division counting results of all 8 resistant samples. FIGS. 7E-7G show comparison of a reference method and digital AST for susceptibility determinations with 30 min, 45 min, and 60 min detection. FIG. 7H show digital AST accuracy over detection time.

### DETAILED DESCRIPTION

This disclosure describes rapid antimicrobial susceptibility testing (AST) systems and methods that implement large volume scattering imaging (LVSi) for real-time imaging of single cells with sensitivity and precision. These systems and methods work directly on urine samples in glass vials or cuvettes to image, track, and count the individual division events of single bacterial cells in clinical samples. These rapid AST systems and methods can image and count low bacterial concentration samples (e.g., 10⁴ CFU/mL urine samples). To precisely track and count single division events of bacterial cells in the presence of various particles (e.g., crystals and dead skin cells) in a sample, a forward scattering optical imaging configuration and an imaging processing algorithm are implemented.

AST systems and methods described herein provide single cell precision in real-time without DNA primers, reagents, incubation, immobilization, or microfluidics. The elimination of microfluidics and associated pumps and valves simplifies the setup, removes clogging of microfluidic channels by air bubbles and impurities in real urine samples, and allows simultaneous tracking of multiple cells in parallel in free solution.

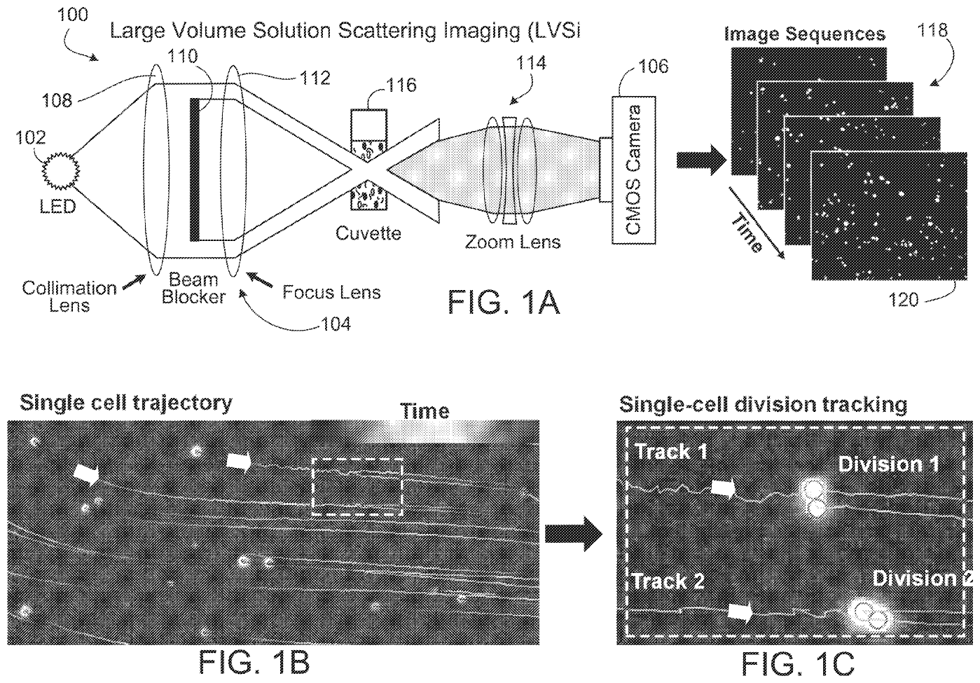

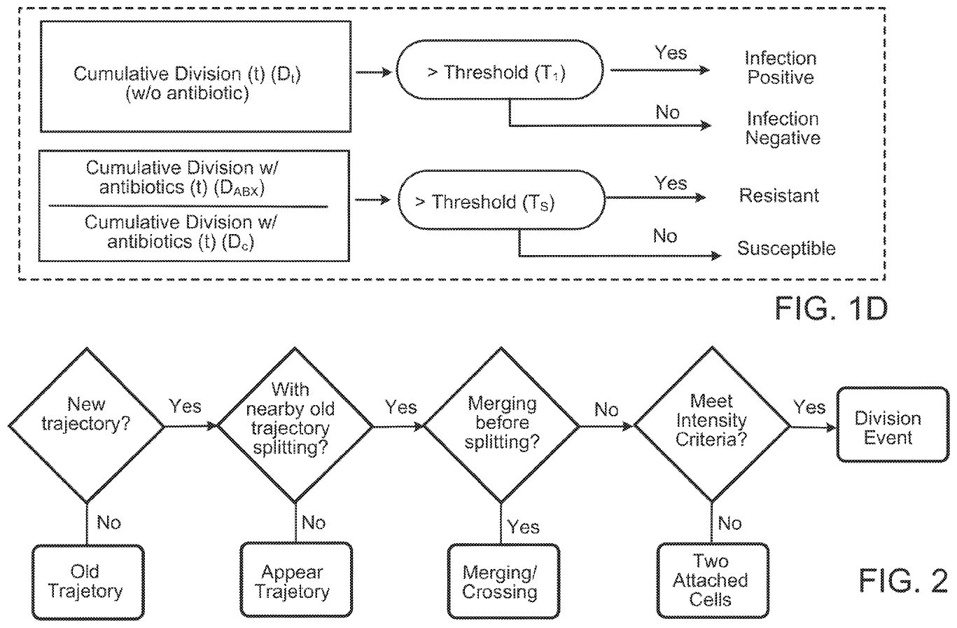

FIG. 1A depicts a large volume scattering imaging (LVSi) system 100 for imaging samples in real time with single cell resolution. System 100 includes light source 102, optics 104, and camera 106. Light source 102 is a light emitting diode (800 mW, 780 nm near infrared (NIR) LED). Optics 104 include collimation lens 108, beam blocker 110, focus lens 112, and zoom lens 114. Sample 116 is positioned between focus lens 112 and zoom lens 114. Sample 116 is contained an optically transparent sample holder (e.g., a cuvette or a vial). Sample 116 is typically a bodily fluid (e.g., urine). Camera 106 is configured to capture a series of images 118 (e.g., a video) of the sample over time. Other implementations may include different types of light sources, different types of optics, different sample holders. With a low magnification factor (0.5-10X), a total image volume (e.g., 1-50 μL), is sufficient to allow hundreds of bacterial cells to be imaged simultaneously at clinically relevant concentrations (about 1×10⁴-1×10⁸ CFU/mL). In image sequences 118, each cell is resolved as a bright cell spot 120.

System 100 typically includes a controller (e.g., a computing device such as a laptop or desk top computer). The controller may be coupled to a network and one or more remote computing devices. The controller can be configured to control light source 102 and camera 106 and analyze images 118.

The controller may be used to implement automated division tracking to extract the trajectory of each individual cell and filter out single-cell division events over time for rapid antibiotic susceptibility determination. To track the real division events, each spot in the series of images or video is connected in time to form single-cell trajectories. For each trajectory, all temporal and spatial information is extracted, including trajectory start time, trajectory duration, trajectory end time, spot location (x, y) in each frame, spot mean intensity in each frame, and so on. With all of this information, the division events were filtered out depicted in the flow chart in FIG. 2. The daughter cell candidate trajectory is understood to be a newly appeared one, and the possible parent trajectory is understood to appear earlier than the daughter cell and be located close to the new trajectory in both time and position. At the start time point of the new trajectory, splitting from a nearby old trajectory is looked for (i.e., one old trajectory splits into two new trajectories). If no splitting is detected, this new trajectory is categorized as an appeared trajectory, which may originate from the z direction. For the trajectory with nearby splitting, a merging event with the old trajectory is looked for before splitting. If present, this merging-splitting is considered as overlapping/crossing event. If absent, the intensity profile of the daughter cell candidate trajectory and parent candidate trajectory is further checked, ensuring the two daughter cell trajectories have similar intensity profiles and the parent trajectory has a higher intensity profile. If the intensity of two daughter cell trajectories are very different, it is likely to be two attached cells or a visual merging event that occurred out of the image view. If the detected splitting satisfies the intensity criteria, it is determined to be a valid division event.

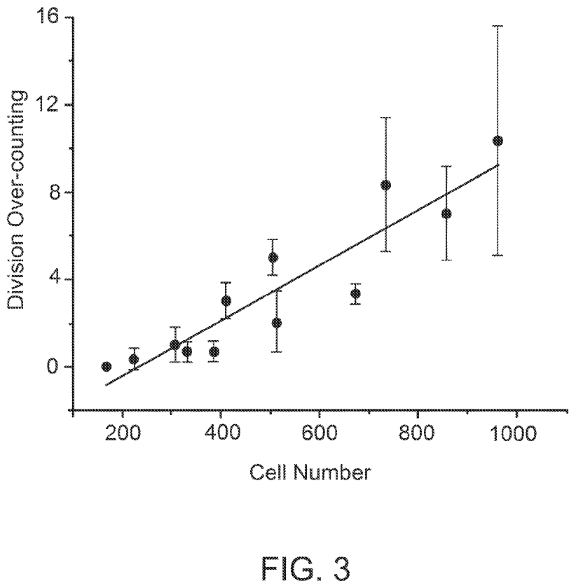

To estimate the single-cell division tracking accuracy, calibration is performed with the heat-deactivated E. coli cells, in which there should be no real growth associated division event. The division events tracked are called division over-counting for final division calibration. With different numbers of E. coli cells, the division over-counting result is shown in FIG. 3, which increases with increasing cell number in the image. When the cell number in view is below 500 (˜1×10⁵ CFU/mL), the division over-counting is small. When the cell number increases, the possibilities of overlapping/crossing increases. If two similar cells merged together outside of the image view and split inside the view, filtering is difficult to achieve. Therefore, to remove the division over-counting effects, the final division results are calibrated by subtracting the division over-counting number.

The automated division tracking algorithm includes one or more of the following operations. In one operation, common background noise and image drift are corrected with temporal local minimal subtraction to improve the image contrast for detection of cell spots. In another operation, a Laplace of Gaussian (LOG) filter is used to detect individual cells. In another operation, directional linking of cell spots in adjacent frames is performed using a Karman algorithm to obtain single cell tracking trajectories of cells 120, such as those depicted in FIG. 1B. To detect the division events, a division filter is applied to identify the events that split a parent cell into two child cells. Two representative events of single-cell division tracking are shown in FIG. 1C, in which parent cells 120 are split into child cells 122.

FIG. 1D depicts clinical decision determination based on detected division events. Dₜ is the cumulative division at time t, while D_{ABX} and D_{C} represent the cumulative division at time tin samples with and without antibiotics. To assess the presence of infection, the single-cell division events are tracked in videos or image sequences obtained over time (e.g., 60 minutes) by system 100. In one example, single-cell division events are tracked in 5-min videos for 60 min. The cumulative division (division events integration, D_{C}) in a control sample without antibiotics (w/o ABX) at the 60 min point is used for infection detection. As depicted in FIG. 1D, if the cumulative division is above the selected infection threshold (D_{C}>T_{I}), the sample is determined to be infection positive. Otherwise, the sample is determined to be infection negative. For antibiotic susceptibility determination, the cumulative divisions in a control sample (w/o ABX, D_{C}) and antibiotic-treated sample (w/ABX, D_{ABX}) are used. Sample pairs that yield a D_{ABX}/D_{C} ratio above a selected susceptibility threshold (T_{S}) are called resistant, and samples with D_{ABX}/D_{C}<T_{S} are called susceptible. D_{ABX}/D_{C}<T_{S} is understood to mean that cell division is slowed or halted in the antibiotic-treated (w/ABX) sample, indicating that the sample is susceptible to that antibiotic dose. D_{ABX}/D_{C}>T_{S} is understood to mean that cell division continued in both the control (w/o ABX) and antibiotic-treated (w/ABX) samples with similar rate, indicating that the sample is resistant to that antibiotic dose.

In an exemplary process, a sample is collected from a subject (e.g., a human) and provided to an imaging apparatus (e.g., system 100 of FIG. 1A). In some cases, the sample is a bodily fluid (e.g., urine). A volume of the sample is typically in a range of 1 μL to 50 μL. The sample may be filtered, diluted, combined with a culture medium, or any combination thereof. However, the sample is not subjected to a conventional culturing process. A multiplicity of images of the sample are collected over time (e.g, with a system such as that described in FIG. 1A), and a trajectory of each bacterial cell in the sample is assessed. Assessing the trajectory of each bacterial cell in the sample includes monitoring a position of each bacterial cell in a sequence of images. Based on the trajectory of each bacterial cell in the sample, a number of bacterial cell divisions that occur in the sample during the length of time is assessed.

An infection threshold is defined as a number of cell divisions, and the number of bacterial cell divisions that occur in the sample during the length of time is compared with the infection threshold. The sample is identified as infection positive if the number of bacterial cell divisions that occur in the sample during the length of time exceeds the infection threshold. The sample is identified as infection negative if the infection threshold (e.g., 2 to 10 cell divisions) exceeds the number of bacterial cell divisions that occur in the sample during the length of time (e.g., between 20 minutes and 120 minutes or between 30 minutes and 60 minutes). Based on the number of bacterial cell divisions, an appropriate antibiotic may be administered to a subject

Results from a first sample that includes an antibiotic may be compared with a second sample from the same source that is free of antibiotic. A ratio of the first number of bacterial cell divisions to the second number of bacterial cell divisions can be assessed. A susceptibility threshold may be defined (e.g., 0.4 to 0.6, corresponding to inhibition of 40% to 60% of the bacterial cells) and compared to the ratio. The first sample may be identified as resistant to the antibiotic if the susceptibility ratio exceeds the threshold. The first sample may be identified as susceptible to the antibiotic if the susceptibility threshold exceeds the ratio.

In the examples below, a LVSi technique is used for detection of bacteria and determination of antimicrobial susceptibility directly in a real sample. By tracking the single-cell division events, growth of the viable cells is quantified with high sensitivity in a short time. For pure E. coli samples without sub-culture, direct AST with the cells from stationary phase was achieved in 60 minutes. Results revealed the variability in the growth rate of cells from different populations, demonstrating the existence of the persistent cells with the presence of antibiotics. The method allows single cell detection capability, enabling the study of heterogeneity of cell response to antibiotics and the antibiotic resistance evolution. For real samples, the technique was applied to 60 clinical urine samples and predicted 97% of the bacterial existence for the infection positive samples with 60 min. AST was also performed on these patient samples with ciprofloxacin, and achieved 100% categorical agreements within 60 min (sample-to-results).

The performance compares well with the existing culturing-based commercial technologies. The technique can test raw clinical samples without enrichment or culturing, and track the division events of individual viable bacterial cells in real time, which simplifies the testing procedures, improves the precision, and shortens the turnaround time from sample receipt to result determination. As the division tracking quantifies the bacterial cell growth, which is a universal phenotypic feature for AST, this technique is applicable to a wide range of bacteria.

### EXAMPLES

E. coli ATCC 25922 were purchased from American Type Culture Collection (ATCC) and stored at −80° C. in 5% glycerol. Antibiotics, including ciprofloxacin and ampicillin were purchased from Sigma-Aldrich. The antibiotic powders were stored in the dark at 2 to 8° C. Frozen E. coli strains were thawed, and 50 μL of the cells were cultured in 5 mL of Luria-Bertani (LB) medium (per liter: 10 g peptone 140, 5 g yeast extract, and 5 g sodium chloride) at 37° C. and 150 rpm for 15 h. Then the overnight cultured E. coli was inoculated into LB broth directly. After dilution to appropriate cell concentrations, antibiotics were added to the E. coli suspensions.

De-identified clinical urine samples were obtained from the clinical microbiology laboratory of Mayo Clinic Hospital Arizona. Clinical samples were transported in an ice box and kept at 4° C. after receiving. The refrigerated urine samples were pre-warmed for 30 min at 37° C. before use. Then, the urine samples were passed through a 5 μm filter to remove the large substances and diluted with LB broth to a concentration of ˜2×10⁵ CFU/mL. The diluted clinical urine samples (100 μL) were added to 96-well microtiter plates (Falcon, BD Biosciences) preloaded with LB broth (100 μL) with and without ciprofloxacin (2 μg/mL, final concentrations). After full mixing, 70 μL samples were transferred to cuvettes (Uvette, Eppendorf, Germany), and subjected to LVSi. A total of 60 urine samples with blind pathogens were tested using both optical division tracking and parallel validating plating. The results were compared with clinical microbiology culture results.

The dual channel LVSi system depicted in FIG. 1A was used, with two 800 mW, 780 nm infrared (IR) LEDs (M780LP1, Thorlabs, Inc., USA), each with collimating and focusing lenses and a central blocking aperture to focus a ring-shaped illumination through the sample or the reference cuvettes. Wide-view and deep field depth scattering images were recorded by two CMOS cameras (BFS-U3-16S2M-CS, Point Grey Research Inc., Canada) at 10 fps through two 2×variable zoom lens (NAVITAR 12×, Navitar, USA), for the sample and reference cuvettes, respectively. The image volume was determined by the viewing size and focal depth of the optics. For these experiments, the viewing area of 2.5 mm×1.9 mm×1.0 mm was equivalent to 4.8 μL at 2.0× magnifying power. The imaging system was enclosed in a thermally isolated housing with a controlled temperature (37° C.).

Individual cells recorded by LVSi are resolved as bright spots. Before division tracking, each spot was detected with a Laplacian of Gaussian (LoG) filter with defined radius and threshold. Then, the spots from adjacent time frames were connected with a Karman filter for directional linking. So each bright spot became a single-cell trajectory. To track the division events, the newly appeared cell trajectories (those from the image edges excluded) were first filtered out as the child trajectory candidates, and for each child trajectory candidate, the nearby old trajectories (appeared before the child trajectory candidate) were checked. If there was a close-by old trajectory split at the start time of the child trajectory candidate, a potential division event was tracked. To filter out the merge/crossing induced splitting, the spots merging events were also checked and were filtered out from the potential division events. Finally, an intensity filter was used to evaluate the remaining division events, ensuring the parent cell intensity is about the summation of the two divided child ones, and the two child ones are similar in size.

To validate that the tracked division events were due to real cell growth, rather than an artifact of the particle merging or crossing, a division over-counting calibration test was designed. To rule out the real division events, the E. coli cells were heated at ˜65° C. for 15 min. Then, each 5-min video of the heated E. coli cells with different concentrations (2.0×10⁴˜2.0×10⁵ CFU/mL, corresponding to 100-1000 spots in image view) were analyzed for single-cell division tracking. Each concentration test was repeated three times. Then, the division over-counting calibration curve was extracted from the tracking results. The division over-counting is less an issue when the cell number is below 500, corresponding to the bacterial concentration of about 1.0×10⁵ CFU/mL. When the cell number is above 500, some miss-counting of division events occurred. To rule out the cell density induced division over-counting, the final division events in all clinical samples were calibrated by subtracting the cell density associated over counted division events.

The statistical error of the division tracking was estimated by the mentioned division over-counting calibration. The final division events were calibrated by subtracting the fitted mean value of the division over-counting, in which the averaged standard error of the mean is calculated to be ˜1 in every 5-min video. To establish a 95% confidence interval, the error was multiplied by 2. For a 60 min detection, the cumulative standard error of the mean is ˜24. Since standard error of the mean in each 5-min is random, the final statistic error of the tracking was estimated by N^{1/2}, where N is the cumulative standard error of the mean. The final threshold for infection identification (T_{I}) was set to be 5 with a 95% confidence for the calibration. The susceptible threshold (T_{S}) was set to be 0.5, corresponding to 50% of the growth inhibition in the antibiotic-treated samples.

Pure E. coli samples were used to establish a single-cell division tracking method. AST was performed directly from the stationary phase bacteria without culturing. The culture-independent capability was tested to mimic clinical urine samples in which the bacteria are likely in stationary phase due to environmental change and lack of nutrients. The pure E. coli samples, in which most of the cells were in stationary phase, were directly diluted into two equal volumes of culture medium (LB broth) without and with antibiotics at standard breakpoint concentration. The breakpoint concentration is the concentration of an antibiotic that defines whether a species of bacteria is susceptible or resistant to the antibiotic. If the minimum inhibitory concentration (MIC) (the lowest concentration of an antibiotic required to inhibit growth of an organism) is less than or equal to the susceptibility breakpoint, the bacteria is considered susceptible to the antibiotic. If the MIC is greater than this value, the bacteria is considered intermediate or resistant to the antibiotic. The diluted bacteria concentration is ˜1×10⁵ CFU/mL. Then, a diluted sample of 70 μL was transferred to an imaging cuvette for direct LVSi for 60 min at 37° C. Image sequences containing hundreds of bacterial cells were obtained and trunked into 5-min videos. Individual cell division events were tracked in every 5-min video and cumulative division events were counted for the entire 60 min.

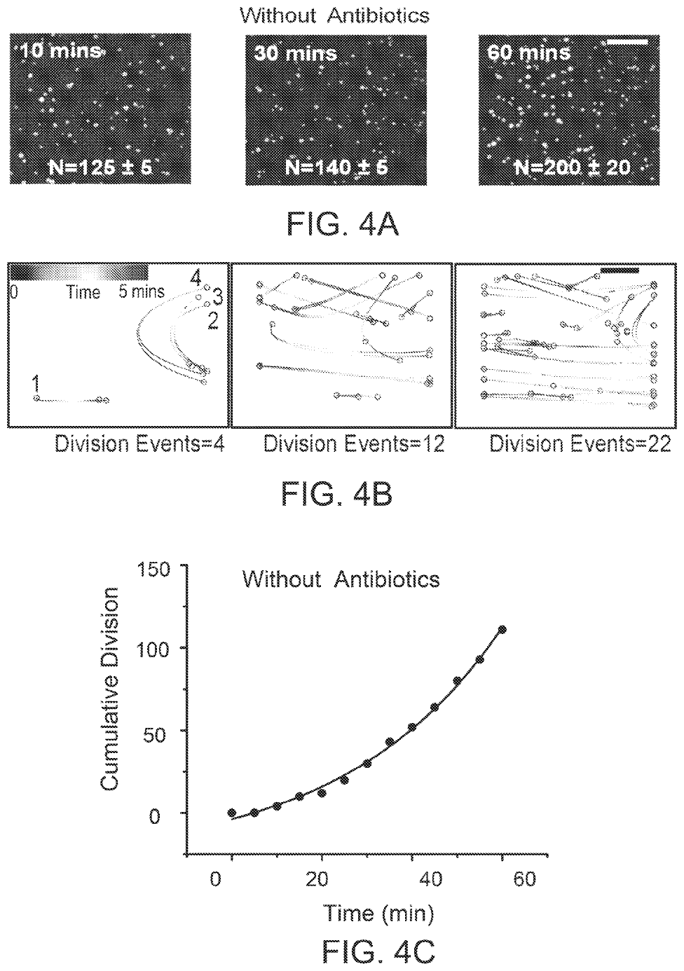

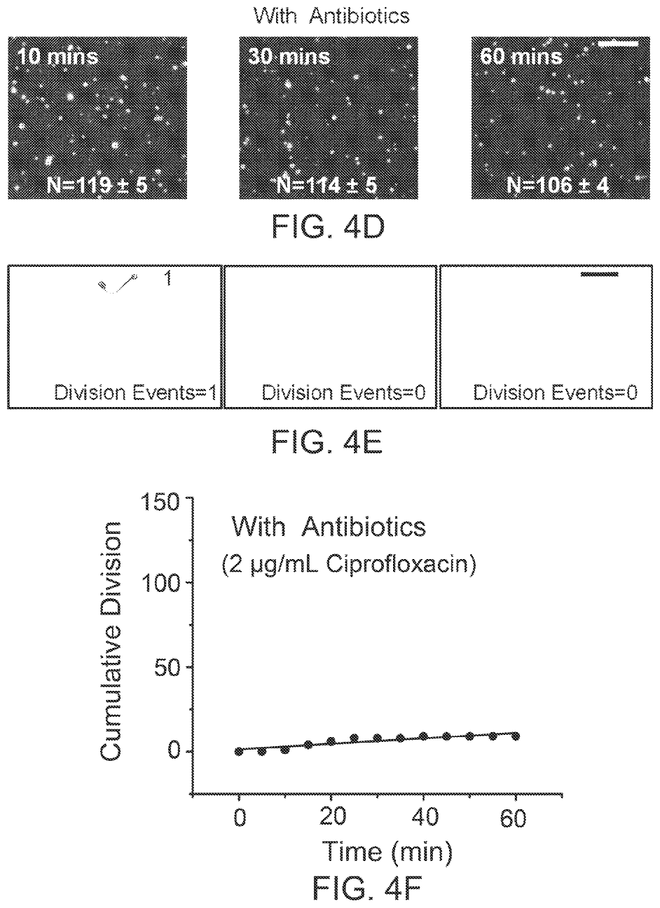

FIGS. 4A-4C and 4D-4F show representative results of digital AST with single-cell division tracking of every 5-min video in control (w/o ABX) and antibiotic-treated (w/ABX, 2 μg/mL ciprofloxacin) tests, respectively. Video snapshots of the control sample in FIG. 4A at 5 min, 30 min, and 60 min show cell spots and the average counting numbers (120±10, 140±5, 200±20, respectively) during 5-min videos (scale bar 400 μm), and the tracked single-cell division events are plotted in FIG. 4B. Each division process starts with a parent cell 120 at the starting position, which moves along the track and splits into two child cells 122. The number of division events is seen to increase with time. By integrating the division events of every 5 min during the whole detection, the cumulative division count can be plotted as in FIG. 4C, which shows exponential growth. In contrast, video snapshots of the antibiotic-treated sample in FIG. 4D at 5 min, 30 min, and 60 min show cell spots and the average counting numbers (118±8, 114±5, 106±4, respectively) during a 5-min video (scale bar 400 μm). FIG. 4E shows a single division event from parent cell 120 to child cells 122 tracked in the antibiotic treated (2 μg/mL Ciprofloxacin). FIG. 4F shows no obvious increases in the growth curve compared with the control sample in FIG. 4C. This result shows clearly that tracking of division events can quantify inhibition of bacterial growth in the presence of antibiotic at the breakpoint concentration.

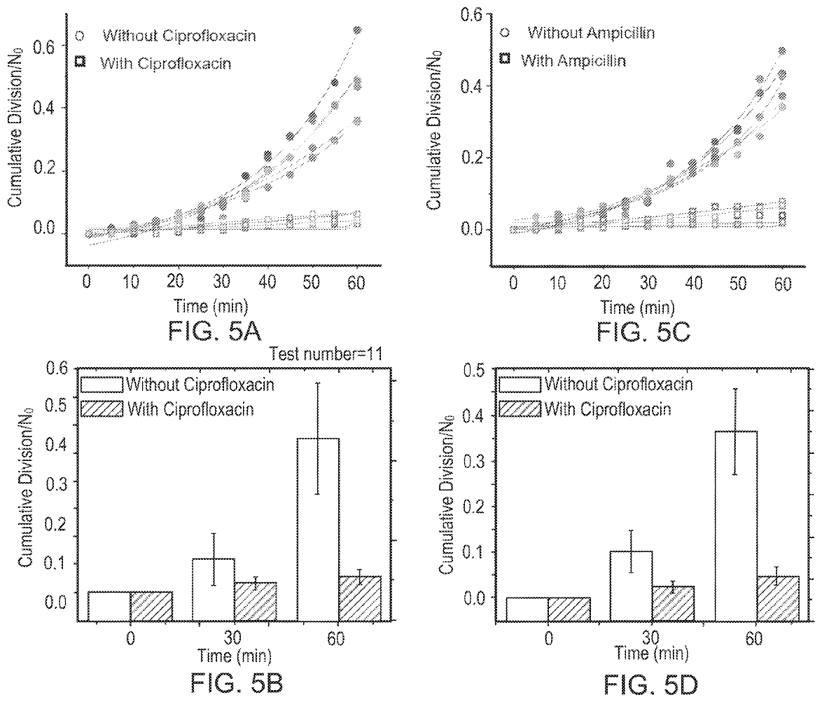

To validate the precision and broader application of the method, more tests with 2 μg/mL ciprofloxacin were performed, along with tests of an additional antibiotic, 16 μg/mL ampicillin. These tests were performed with different batch of E. coli samples. To compare the results from different experiments, the cumulative division events were normalized by the number of initial cells (No), showing the increase in cell growth. For clarity, 5 representative cumulative division tracking results of E. coli samples with and without antibiotics are plotted in FIG. 5A (ciprofloxacin) and FIG. 5C (ampicillin). All control experiments show exponential growth in cumulative division counting with variation in growth rate observed for different samples, which is believed to depend on the initial state of the bacterial cells and the environmental control. With the presence of antibiotics, all samples showed clear inhibition in bacterial cell growth, demonstrating the capability of division tracking for susceptibility testing of antibiotics with different mechanisms of action. Ciprofloxacin directly stops cell division by inhibiting DNA replication, while ampicillin inhibits bacterial cell wall synthesis. The statistical results of 11 tests in FIGS. 5B and 5D show the cumulative division events at different time points (0 min, 30 min and 60 min) (p<0.001). With the time increase, more divisions were detected in the control samples, and a significant difference between control and the antibiotic-treated sample was observed by the time of 60 min.

With the single-cell division tracking capability, some cells were still observed to divide in the presence of antibiotics, showing the cell-cell heterogeneity within a sample. Since these persistent cells in the antibiotics may eventually develop to a resistant strain, the digital counting method described herein provides a capability for early warning on potential drug resistance or tracking the progress of resistance development.

The E. coli samples from different batches were used to evaluate the influence of the sample variability on the robustness of the digital AST method. The effects of the bacterial initial status on the growth curve were observed. The samples were directly tested without sub-culture, with most of the cells are in stationary phase. For comparison, the susceptibility tests were also performed with E. coli from log phase (with 2 hr sub-culture). In log phase, sufficient division events occurred sooner and the total AST time was reduced to about 30 min. These results demonstrated that the cell status in the sample affect the total detection time. Effects of the environmental control were also examined. Comparison of the results from the optical system, and incubator with and without shaking suggests that continued measurement does not have a significant impact on the growth of the bacteria. To cross validate the division tracking accuracy, traditional plating detections were performed simultaneously on the same samples to verify the cell growth at each time point, and the results were consistent with the division tracking ones.

To demonstrate the capability of the digital AST method, the optical division tracking method was used with clinical urine samples for urinary tract infection (UTI) diagnosis and susceptibility determination. Digital AST was implemented directly with 60 de-identified clinical urine samples with blinded pathogens, including negative samples. Before detection, a simple filter (5 μm) was performed to remove the large substances. Then, the clinical urine samples were diluted with LB medium to a concentration around ˜1×10⁵ CFU/mL and imaged for 60 min in a setup such as that depicted in FIG. 1A. Infection detection and AST results were determined by tracking the single-cell division events in control and antibiotic-treated samples. A parallel plating validation was performed.

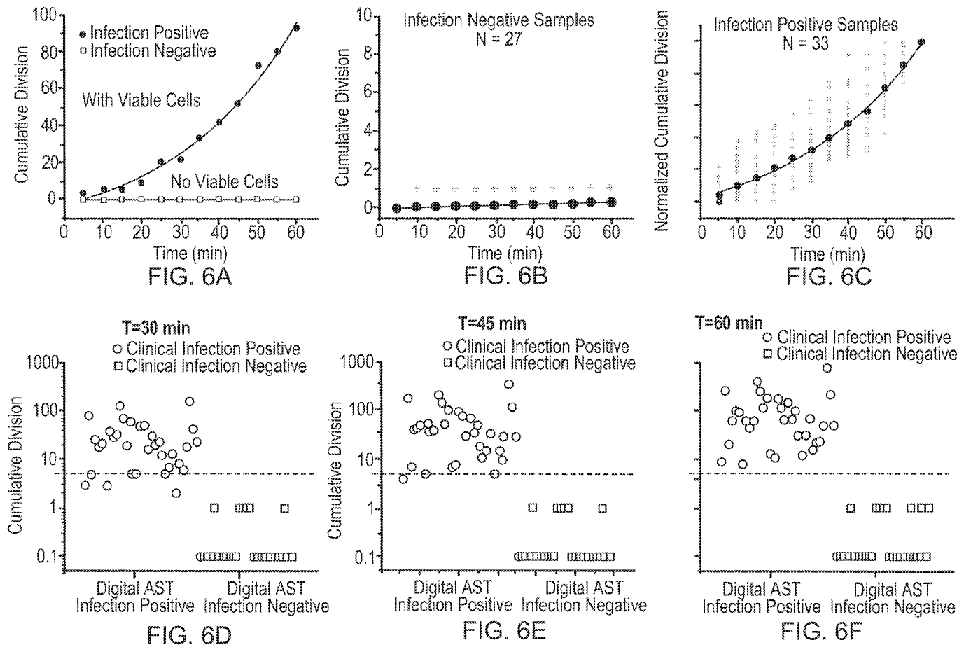

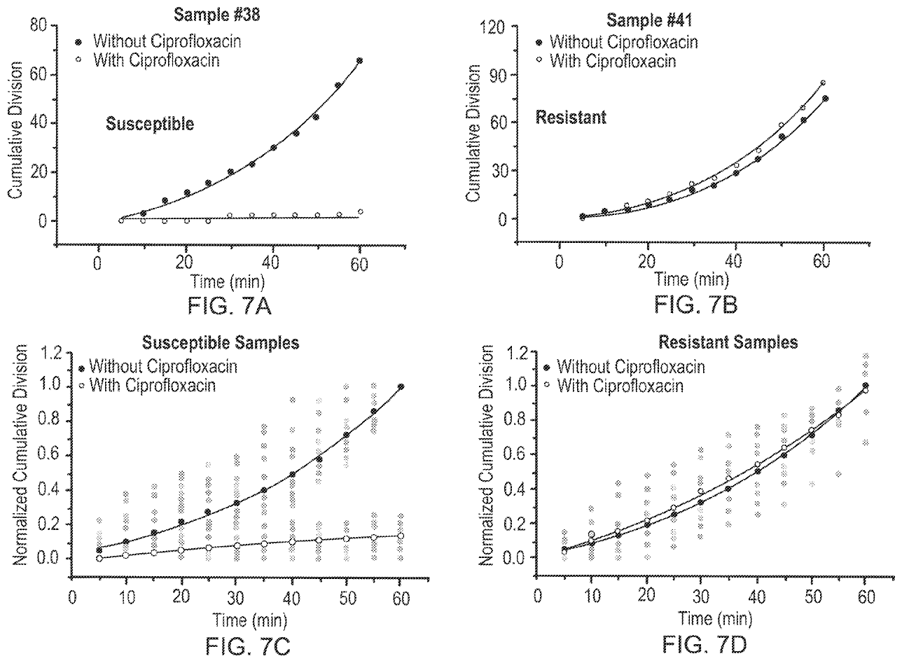

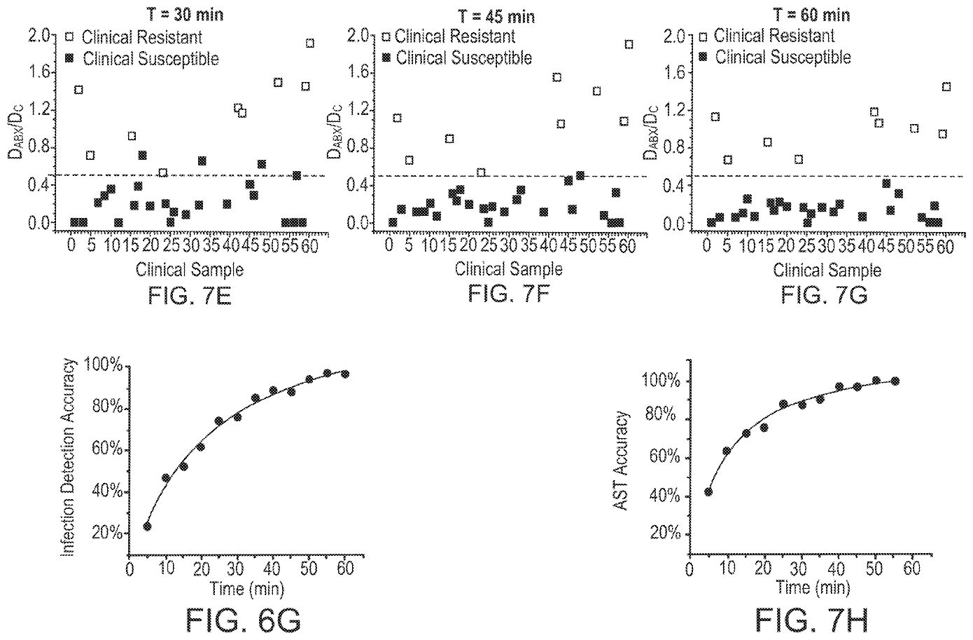

FIGS. 6A-6G show the results of rapid infection detection with 60 clinical urine samples. FIG. 6A shows a representative division tracking result 400 of one infection negative sample. In this sample, there were no viable cells, and no division events were tracked. In contrast, a representative division tracking result 402 of one infection positive sample, shows growth in bacterial cells grow with time, and division events were found. By tracking of the single-cell division events of all 60 clinical samples without antibiotic treatment, the cumulative divisions at 60 min were extracted for infection determination. Due to the high density of particles in clinical samples, the final division results were calibrated to remove the cell density induced division over-counting. The infection threshold was determined to be 5. FIGS. 6B and 6C show cumulative division of infection negative samples and normalized cumulative division of infection positive samples, respectively. By comparing the final cumulative divisions with the infection threshold, 30 samples were determined to be infection negative, where in most of these negative samples the cumulative events were 0, and others smaller than 5. 30 samples were determined to be infection positive, with division counting variations ranging from 10 to several hundreds.

To explore the infection detection accuracy over time, comparison of a reference method (BD Phoenix) and digital AST at the time points of 30 min, 45 min and 60 min are shown in FIGS. 6D-6F, respectively. For the infection negative samples, all 0 values are changed to 0.1 for log scale plot. With the 30 min detection, most of the infection positive samples show measurable growth, with a detection accuracy of ˜90%. With 45 min detection, the detection accuracy increased to 93%. At 60 min, all infection positive samples were determined except for one false positive, showing an accuracy of ˜97%. FIG. 6G, a plot of infection detection accuracy over 60 min, shows a hyperbolic increase over time.

Digital AST was performed for all of the 30 infection positive samples. The susceptibility profiles were determined by comparing the calibrated cumulative division in control (D_{C}) and antibiotic (ciprofloxacin) treated tests (D_{ABX}). For a susceptible sample, such as that depicted in FIG. 7A, bacteria in the control grew exponentially over time, while the cell growth in the antibiotic-treated sample was inhibited. Thus, cumulative divisions in the control sample increased over time, while few division events were detected in the antibiotic-treated sample. In contrast, for a resistant sample, such as that depicted in FIG. 7B, the bacterial cells in the control and antibiotic-treated samples grew exponentially with a similar rate, as shown by the similar cumulative division counts.

For these samples, the susceptibility threshold (T_{S}) was set to be 0.5, which indicates 50% of the cell growth inhibition. By comparing the D_{ABX}/D_{C} ratio to the susceptibility threshold, 8 samples were determined to be resistant to ciprofloxacin and the other 22 samples were determined to be susceptible to ciprofloxacin. As shown in FIG. 7C, the growth of the susceptible bacteria (in susceptible samples) under ciprofloxacin was inhibited compared to the control samples. As shown in FIG. 7D, the growth of the resistant bacteria (in resistant samples) under ciprofloxacin treatment was indistinguishable from the control. These results were in 100% agreement with results from both clinical microbiology testing and the parallel plating validation. To explore the AST accuracy over time, FIGS. 7E-7G show comparison of a reference method (BD Phoenix) and digital AST plotted at the time points of 30 min, 45 min and 60 min, respectively. With the 30 min detection, 3 susceptible samples were in the resistant zone with another 2 samples on the border line, demonstrating a category accuracy of ˜87%, while the 45 min detection increased the category accuracy to 94%. At 60 min, all susceptibility profiles were corrected determined, showing an accuracy of 100%. FIG. 7H shows a hyperbolic increase in AST accuracy over the 60 min.

Thus, LVSi with single-cell division tracking technology that rapidly detects the existence of bacteria and determines the antibiotic susceptibility was demonstrated. Use of a large image volume allowed use of a real sample directly without further enrichment. The single-cell division tracking and counting provided high sensitivity for rapid cell growth quantification. In clinical urine samples, there are particle impurities, some of which tend to precipitate during the test. Thus, in a spot counting method, the cell growth induced spot increases will be compromised by the sedimentation of impurities. The division tracking show advantages for measuring viable cell grow, reducing the total detection time compared to the spot counting results in clinical sample.

For accurate division tracking, one variation here is the final sample concentration. The single-cell division is based on the accurate particle detection and trajectory linking. When the particle density is too high, linkages may be assessed incorrectly. Based on the current linking algorithm, the linking accuracy is affected when the particle number is above 1000, which corresponds to the particle density of about 2.0×10⁵/mL. Therefore, for optimal division tracking, the clinical sample is diluted to a concentration of about 1×10⁵ CFU/mL before testing. Since there are urine particles in the samples, not all spots in the video are viable bacterial cells. To extract the dynamic range of the digital AST method, a minimal viable bacteria number needed for 60-min AST is assumed to be 50. When the viable bacteria number is below 50, a longer time is needed for digital AST. Then, the range of the viable bacterial cells in the video is 50-1000, and the dynamic range of the method is derived to be 1×10⁴-2.0×10⁵ CFU/mL.

Sample dilution is another factor for consideration. For optimal division tracking, clinical samples were diluted to make sure the total particle number in the video was below 1000. However, in some samples, the raw bacteria concentration is in the lower clinical range (between 1.0×10⁴ to 1.0×10⁵ CFU/mL). With an extra dilution step, these samples are likely to be over diluted, leading to a very small number of viable bacteria in the imaging system. A false negative result may be due to over dilution, with no viable bacterial cells after dilution based on the validation plating results. To reduce the influence of dilution on false infection negative results, the sample preparation process can be optimized to remove extra urine particles, the image volume can be increased, and the division tracking algorithm can be modified.

Additional details are provided in Exhibit 1.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

1. A method of detecting single bacterial cells in a sample, the method comprising:
collecting, from a sample provided to an imaging apparatus, a multiplicity of images of the sample over a length of time;
assessing a trajectory of each bacterial cell in the sample; and
assessing, based on the trajectory of each bacterial cell in the sample, a number of bacterial cell divisions that occur in the sample during the length of time.

2. The method of claim 1, further comprising providing the sample to the imaging apparatus; and/or further comprising collecting the sample from a subject.

3. (canceled)

4. The method of claim 2, wherein the sample comprises a bodily fluid from a subject.

5. The method of claim 4, wherein the sample comprises urine.

6. The method of claim 2, further comprising combining the sample with a culture medium; further comprising diluting the sample; and/or further comprising filtering the sample.

7. (canceled)

8. (canceled)

9. The method of claim 1, further comprising:
defining an infection threshold as a number of cell divisions; and
comparing the number of bacterial cell divisions that occur in the sample during the length of time with the infection threshold.

10. The method of claim 9, further comprising identifying the sample as infection positive if the number of bacterial cell divisions that occur in the sample during the length of time exceeds the infection threshold; or further comprising identifying the sample as infection negative if the infection threshold exceeds the number of bacterial cell divisions that occur in the sample during the length of time.

11. (canceled)

12. The method of claim 9, wherein the infection threshold is between 2 to 10 cell divisions.

13. The method of claim 1, wherein the sample is a first sample, the length of time is a first length of time, and the number of bacterial cell divisions that occur in the sample during the first length of time is a first number of bacterial cell divisions, and further comprising:
collecting, from a second sample provided to the imaging apparatus, a multiplicity of images of the second sample over a second length of time;
assessing a trajectory of each bacterial cell in the second sample; and
assessing, based on the trajectory of each bacterial cell in the second sample, a second number of bacterial cell divisions that occur in the second sample during the second length of time.

14. The method of claim 13, wherein the first sample and the second sample are obtained from a common source; and/or wherein the first sample comprises an antibiotic and the second sample is free of added antibiotic.

15. (canceled)

16. The method of claim 14, further comprising assessing a ratio of the first number of bacterial cell divisions to the second number of bacterial cell divisions.

17. The method of claim 16, further comprising:
defining a susceptibility threshold; and
comparing the ratio to the susceptibility threshold.

18. The method of claim 17, further comprising identifying the first sample as resistant to the antibiotic if the susceptibility ratio exceeds the threshold; or further comprising identifying the first sample as susceptible to the antibiotic if the susceptibility threshold exceeds the ratio.

19. (canceled)

20. The method of claim 17, wherein the susceptibility threshold is in a range of 0.4 to 0.6, corresponding to inhibition of 40% to 60% of the bacterial cells, respectively.

21. The method of claim 1, wherein a volume of the sample is in a range of 1 μL to 50 μL; wherein a number of particles in the sample is less than about 2×10⁵ particles/mL; wherein assessing the trajectory of each bacterial cell in the sample comprises monitoring a position of each bacterial cell in a sequence of images; and/or wherein a magnification of the imaging apparatus is in a range of 0.5-10×.

22.-24. (canceled)

25. The method of claim 1, wherein collecting the multiplicity of images comprises irradiating the sample with infrared light.

26. (canceled)

27. (canceled)

28. The method of claim 1, wherein the sample is uncultured.

29. The method of claim 1, further comprising, based on the number of bacterial cell divisions, administering an antibiotic to a subject.

30. (canceled)

31. The method of claim 1, wherein the length of time is in a range between 20 minutes and 120 minutes, or between 30 minutes and 60 minutes.

32. A system comprising:
a light source;
optics configured to focus light from the light source on a liquid sample in a container;
an imaging device; and
a controller operably coupled to the light source and the imaging device and configured to:
initiate collection of a series of images of the liquid sample over a length of time;
based on the images, assess a trajectory of each bacterial cell in the sample; and
assess, based on the trajectory of each bacterial cell in the sample, a number of bacterial cell divisions that occur in the sample during the length of time.
